(19) United States
(12) Patent Application Publication
(10) Pub. No.: US 2013/0030324 A1
(43) Pub. Date: Jan. 31, 2013

(54) MALIGNANCY REMOVAL WITH TISSUE ADHESIVE SECURED STRUCTURE

(71) Applicant: Bruno Lowinger, Ann Arbor, MI (US)

(72) Inventor: Bruno Lowinger, Ann Arbor, MI (US)

(21) Appl. No.: 13/638,612

(22) PCT Filed: Apr. 1, 2011
(86) PCT No.: PCT/US11/30891

§ 371 Date: Sep. 30, 2012

Related U.S. Application Data
(60) Provisional application No. 61/320,078, filed on Apr. 1, 2010.

(51) Int. Cl. A61B 10/02; A61B 17/3205

(52) U.S. Cl. 600/567; 606/110

(57) ABSTRACT

Processes of preventing tumor cell mestastases induced by surgical procedures such as resection or biopsy are provided. Some embodiments include applying a tissue adhesive to a structure that is then adhered to tissue providing a tensioning surface for exposing normal tissue for cutting thereby preventing contact of a surgical instrument with tumor tissue. Methods of preventing tumor cell metastasis during or following biopsy are also provided whereby an adhesive alone or optionally along with a structure occupy a track left by a biopsy needle and provide a physical barrier between tumor tissue and normal tissue. The inventive processes improve patient outcome by reducing surgically induced tumor cell metastases.

### CROSS REFERENCE TO RELATED APPLICATIONS

This application claims priority from U.S. Provisional Application No. 61/320,078 filed Apr. 1, 2010, the entire contents of which are incorporated herein by reference.

### FIELD OF THE INVENTION

The present invention generally relates to medical procedures involving malignant tissue, and in particular, relates to using a tissue adhesive to secure a structure in contact with a tumor to prevent malignant cell transference during procedural manipulation.

### BACKGROUND OF THE INVENTION

There is a recognition that the surgical procedures associated with resecting cancerous tissue or biopsy carry the prospect of the inadvertent translocation of cancerous cells to other portions of the body with the net result that potential metastases are produced. The development of a secondary malignancy resulting from the currently employed techniques for resection or biopsy requires a patient to undergo additional procedures that can lead to further translocation to surrounding tissue relative to the secondary malignancy.

Evidence of surgically induced tumor metastases are known in the art. For example, studies of the occurrence of colorectal cancer recurrence following surgery suggest that both local and remote recurrences are not uncommon. (van der Bij, G, et al., Annals of Surgery, 2009; 249(5):727-34.) In addition, tumor cells have been found on surgical gloves and instruments following resection procedures. Curran et al, Abstract 401, 4th Int Conf on Head and Neck Cancer, Toronto, Canada, 1996. Several studies of tumor recurrence indicate that the recurrent tumor shares the same clonal origin as the primary tumor. Studies of tumor cell karyotype and fluorescence in situ hybridization (FISH) has demonstrated synchronous primaries and their recurrences may be of monoclonal origin. (Carey et al, Human Pathol, 1995; 26:251-261.) Complete tumor resection using current techniques can therefore lead to tumor cell translocation which may cause a localized tumor to become mobile.

Coating a tumor with a tissue adhesive has attempted to address cancer cell transference problems as detailed in U.S. Pat. No. 6,341,608. However, while a thin coating of adhesive provides a physical barrier to cancer cell transference, the adhesive coating is itself structurally too weak to support manipulation and is susceptible to damage allowing for tumor cell migration through the tissue adhesive barrier.

Thus, there exists a need for a barrier to isolate malignant tissue from normal tissue during and immediately after surgery and biopsy procedures.

### SUMMARY OF THE INVENTION

The following summary of the invention is provided to facilitate an understanding of some of the innovative features unique to the present invention and is not intended to be a full description. A full appreciation of the various aspects of the invention can be gained by taking the entire specification, claims, drawings, and abstract as a whole.

Processes for malignancy removal or biopsy are provided that reduce or eliminate exposure of tumor cells to normal surrounding tissue or to the bloodstream during or following surgical procedures. Processes of tumor cell removal include applying a tissue adhesive in simultaneous contact between tissue and a surface of an overlying structure wherein the tissue is malignant tissue, or the tissue is non-malignant tissue within 2 centimeters of a malignancy, allowing sufficient time for the adhesive to cure forming a bond between the structure and the tissue; and cutting into a margin of normal tissue surrounding the malignancy outside said structure.

A structure when adhered to a tissue is capable of providing a surface for assisting in excising a tissue. An attachment for tensioning the structure during cutting is optionally provided. The attachment is optionally a suture, a flap formed by a crease in said structure, or a portion of a rigid or flexible structure that a surgeon can apply excisive force to such as by connection to a syringe, a glove, or other instrument. A structure optionally includes collagen. In some embodiments, a structure is in the form of a sheet. A structure optionally has a curvilinear outer shape such as in the form of a circle or oval and defining a margin.

A dam is optionally provided around the malignancy prior to applying the adhesive. In some embodiments, a structure is a casting form with sufficient rigidity to serve both as a structure and a dam.

An adhesive is optionally a one-part or two-part adhesive. Two-part adhesive optionally includes a cross-linkable protein, and a cross-linking agent solution including an aldehyde and an amino acid containing species reactive with the aldehyde. In some embodiments, the aldehyde and amino acid containing species are present in a ratio between 20:1 and 1:1. Optionally, the protein and cross-linking agent are present in a ratio of between 15:1 and 1:1.

In some embodiments, an adhesive, a structure, or both includes a pharmaceutical agent.

Also provided are processes of inhibiting malignant cell transference during a biopsy including applying a tissue adhesive to a track formed by a biopsy needle. The adhesive is optionally applied within 10 seconds of a biopsy needle retracting into a cannula. An adhesive is optionally a one-part or two-part adhesive. Two-part adhesive optionally includes a cross-linkable protein, and a cross-linking agent solution including an aldehyde and an amino acid containing species reactive with the aldehyde. In some embodiments, the aldehyde and amino acid containing species are present in a ratio between 20:1 and 1:1. Optionally, the protein and cross-linking agent are present in a ratio of between 15:1 and 1:1. An adhesive is optionally administered through the channel in a cannula used by a biopsy instrument or through one or more barrels associated with the cannula either integrally or by physical association. In some embodiments, an adhesive is administered only by prior coating onto a structure and administering is therefore coincident with placing the structure in the track.

A flexible structure is optionally placed into the track in contact with the adhesive to form a structure supported adhesive plug. A structure is optionally placed into the track absent prior coating with adhesive or after precoating with adhesive.

In some embodiments, an adhesive, a structure, or both include one or more pharmaceutical agents.

### BRIEF DESCRIPTION OF THE FIGURES

FIGS. 1A-1D depict embodiments of tissue adhesive secured structures intended to provide a barrier against transference of cells from an adhesed malignancy with a patch (1A), a flap containing patch (1B), a casting ring bordering a patch (1C) and a casting dam (1D), the various dimensions of inventive structure being distorted for illustration's sake; and

FIGS. 2A-2D depict the procedural steps in utilizing an inventive tissue adhesive secured structure to inhibit malignant cell transference as part of a biopsy procedure with a cannula being inserted proximal to a suspect mass (2A), a biopsy needle extending through the cannula to biopsy within the suspect mass (2B), a foldable patch coated with tissue adhesive being urged through the cannula (2C) to adhesively secure the biopsy needle path beyond the cannula (2D).

### DESCRIPTION OF EMBODIMENTS OF THE INVENTION

The following description of embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only.

The present invention has utility as a physical barrier to prevent malignant cell transference in the course of a surgical procedure to remove or biopsy suspect tissue. Processes are provided for removal of part or an entire portion of tissue that is malignant, is suspected of being malignant, or has malignant properties. Inventive processes, therefore, have utility for resection of deep tissue or superficial tumors. A process according to the present invention includes applying an adhesive in simultaneous contact between tissue and a surface of an overlying structure wherein the tissue is malignant tissue or non-malignant tissue within 2 centimeters of a malignancy, allowing sufficient time for the adhesive to cure forming a bond between the structure and the tissue, and cutting into a margin of normal tissue surrounding the malignancy outside said structure.

An adhesive as used herein is operable to adhere to a tissue from an organism. As such, the present invention uses a tissue adhesive cross linkable in contact with skin or moist internal tissues, or mucosal membrane. Tissue adhesives have the requirements of cross linking or other polymerization processes to secure a structure in proximity to a suspect malignancy with sufficient force to allow the structure to be pulled or otherwise put under tension to facilitate malignancy excision around a normal cell periphery so as to avoid cutting into the malignancy mass and thereby facilitating transference of cells therefrom to surgical equipment and possibly to secondary tissues.

Illustrative examples of tissue adhesives operative herein illustratively include cyanoacrylates, and those detailed in U.S. Pat. Nos. 7,459,295; 7,351,426;7,141,428; 7,091,015; 7,083,634; 6,939,364; 6,875,427; 6,780,840; 6,773,699; 6,723,114; 6,596,318; 6,565,539; 6,500,427; 6,447,774; 6,310,036; 6,299,631; 6,251,370; 6,234,994; 6,136,341; 6,033,654; 5,980,866; 5,883,078; 5,817,303; 5,665,067; 5,464,471; 5,407,671; 4,909,251; 4,813,928; 4,735,616; 4,631,055; 4,600,574; 4,414,976; 4,377,572; 4,362,567; 4,359,049; and 4,298,598.

In some embodiments, the tissue adhesive is non-necrotic and is of high strength and variable cross-linking timing as detailed in U.S. Pat. No. 7,129,210. In some embodiments, a tissue adhesive includes a cross-linkable protein, and a cross-linking agent solution including an aldehyde and an amino acid containing species reactive with the aldehyde. In some embodiments, the aldehyde and amino acid containing species are present in a ratio between 20:1 and 1:1. In some embodiments, the protein and cross-linking agent are present in a ratio of between 15:1 and 1:1.

A cross-linkable protein according to the present invention is defined herein to include a protein capable of dissolving to form a solution or forming a suspension with a physiologically suitable aqueous solvent. Illustrative cross-linkable proteins include: ovalbumins; serum albumins; recombinantly expressed albumin illustratively expressed in rice such as Oryza sativa; albumin as described by Mawal et al. Biosci Rep., 1987;7(1):1-9, optionally that of GenBank Accession No: BAF12524; and gelatins of human or animal origin from animals illustratively including horse, pig, cow, sheep, kangaroo, chicken, and fish. In some embodiments, a cross-linkable protein is human serum albumin isolated following expression in rice such as Oryza sativa which has the surprising property of differing cross linking relative to other human serum albumin. Suspension of collagen fibers is appreciated to be operative herein as a cross-linkable protein. In some embodiments a cross-linkable protein is albumin derived from a plant source such as rice. Rice albumin It is appreciated that recombinant whole or truncated proteins are operative herein so long as the recombinant proteins remain cross-linkable. Recombinant human serum albumin is operative as a cross linkable protein and the protein is described in U.S. Pat. Nos. 5,633,146; 5,986,062; 5,521,287; and 5,440,018. A recombinant protein is appreciated to lack viral, prion or bacterial contaminants associated with harvested proteins. An albumin operative herein may contain lesser amounts of other proteins or carbohydrates such as those found in blood plasma or elsewhere in a source organism. Human serum albumin is an illustrative cross-linkable protein operative in the present invention as utilized in the context of human tissue repair. It is further appreciated that ultrafiltration or other purification technique as applied to an albumin is successful in reducing the risk of immunological response or infectious agent introduction through the use of the present invention.

To form the first component of an inventive tissue adhesive sealant, a cross-linkable protein is dissolved in water or suspended in water to form a solution containing from 1 to 80 weight percent cross-linkable protein. In some embodiments, serum albumin is used as the cross-linkable protein from 1 to 55 percent by weight. In some embodiments, rice albumin is used as the cross-linkable protein, optionally from 20-65 percent by weight or any value or range therebetween, optionally 30-70 percent by weight, optionally 35-55 percent by weight. In some embodiments, aqueous solution proteins are present from 10 to 55 total weight percent. In some embodiments, aqueous suspension proteins are present from 0.3 to 9 total weight percent. Optionally, the cross-linkable protein is dissolved in an aqueous solution of physiologically acceptable buffer. Optionally, the protein is maintained in a dry or powder form until mixed with the cross-linking agent. Saline is an exemplary physiological buffer. Optionally, a cross-linkable protein solution includes an additive that illustratively includes an electrolyte, a thickener, an anti-microbial, a preservative, and a colorant. An electrolyte additive, if present, is optionally found in an amount that ranges from 0 to 5 total weight percent and illustratively includes sodium chloride, potassium chloride and sodium phosphate. A cross-linkable protein solution thickener according to the present invention is present from 0 to 50 total weight percent. Thickeners operative in the cross-linkable protein solution illustratively include sterilized collagen particulate, implantable grade fibrous materials such as polyamides, fluoropolymers and silk. A thickener in the present invention serves to modify the handling properties of the cross-linkable protein solution as well as to modify the mechanical properties of the resulting tissue adhesive seal. Other optional additives such as an anti-microbial, preservative and a colorant are those conventional to the art and are each present in an amount that typically ranges from 0 to 3 total weight percent. Remington's Pharmaceutical Sciences, 16th Ed., 1980, Mack Publishing Co., Easton, Pa. and in Goodman and Gilman's The Pharmacological Basis of Therapeutics by Hardman and Limbird, 9th Ed., 1996, McGraw-Hill, New York and in The Merck Index: an encyclopedia of chemicals, drugs, and biologicals, 12th Edition, 1996, Merck & Co., Whitehouse Station, N.J. While it is appreciated that the viscosity of a cross-linkable protein solution according to the present invention is controlled through parameters that include cross-linkable protein concentration, the amount and identity of thickener, and the presence of various other additives. A cross-linkable protein solution viscosity is readily tailored to a specific task and has viscosity between that of water and 10,000 centipoise. In some embodiments, a cross-linkable protein solution has a viscosity sufficient to prevent runnage and therefore is generally in a range of between 10 and 1,000 centipoise.

A cross-linking agent solution component that upon combination with the cross-linkable protein solution forms a tissue adhesive includes a multivalent aldehyde and an amino acid containing species reactive therewith. The multivalent aldehyde is optionally a divalent aldehyde having a molecular weight of less than 1,000 Daltons. Optionally, the multivalent aldehyde has a C₀-C₁₆ alkyl or aryl backbone intermediate between two terminal aldehyde groups. Optionally, an aldehyde is a C₃-C₈ linear alkyl dialdehyde. Glutaraldehyde is an illustrative species of linear alkyl dialdehyde. It is appreciated that the introduction of a lesser quantity of a tri- or polyaldehyde with a majority of a dialdehyde creates cross-linkages within the cross-linking agent resulting in modified solution viscosity and final tissue adhesive mechanical properties. Optionally, a tri- or polyaldehyde is present at a stoichiometric molar ratio relative to a dialdehyde of 1:1000-1:30.

An amino acid containing species is reacted with a multivalent aldehyde to form an oligomeric cross-linking agent. The amino acid containing species reactive with the multivalent aldehyde illustratively includes α-amino acids, β-amino acids, dipeptides, polypeptides, proteins, glycoproteins, and combinations thereof. It is appreciated that both d- and l-conformers of a given amino acid are operative herein with the corresponding bioabsorbability associated with each conformer. It is appreciated that an amino acid containing species according to the present invention includes salts, esters and derivatized forms thereof. Additionally, where the amino acid is a β-amino acid, the resulting adhesive is comparatively resistive to bioabsorption. Derivatives to an amino acid containing species according to the present invention include: solvation enhancing moieties such as hydroxyls, thiols, sulfonyls, halos; antibiotics; radioisotopes; magnetic markers; and antibodies. Amino acids optionally include: acidics: glutamic and aspartic acid; aliphatics: alanine, valine, leucine and isoleucine; and amides glutamine and asparagine. In some embodiments, an amino acid containing species is shown in Formula I:

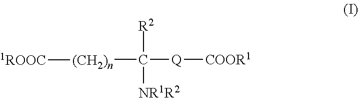

where Q is CH₂ or a nullity, R¹ is independently in each occurrence H, Na, K, C₂-C₆ alkyl; R² is independently H, C₁-C₂₀ alkyl group, a C₀-C₄ alkyl group having a substituent selected sulfonate, carboxylate, hydroxyl, quaternary amines, a radio isotopic ion, a magnetically detectable ion, an antibiotic moiety and an antibody; and n is an integer between 1 and 6 inclusive; hydrohalide salts thereof; and combinations thereof.

Some embodiments of the amino acid containing species of Formula I are L-glutamic acid, L-glutamic acid hydrochloride, sodium L-glutamate, potassium L-glutamate, monosodium L-glutamate, monopotassium L-glutamate, L-aspartic acid, L-aspartic acid hydrochloride, sodium L-aspartate, potassium L-aspartate, monosodium L-aspartate, and monopotassium L-aspartate, and combinations thereof. L-glutamic acid and L-aspartic acid are may be used owing to the resulting cross-linking efficacy. It is appreciated that monosodium L-glutamate, L-glutamic acid hydrochloride, monopotassium L-glutamate, monosodium L-aspartate, L-aspartic acid hydrochloride, and monopotassium L-aspartate into a cross-linking solution for a longer period of time yield similarly effective cross-linking solutions relative to L-glutamic acid.

According to the present invention, the amino acid containing species is present in the cross-linking agent solution in an amount such that the molar ratio of aldehyde moieties to amino acid or peptide subunits is between 20:1 and 1:1. It is noted that within this ratio range, an increase in amino acid containing species generally tends to increase the ultimate adhesive and cohesive strengths of the cured tissue adhesive sealant. Optionally, the aldehyde moieties to amino acid or peptide subunits molar ratio is between 10:1 and 4:1. Optionally, the ratio is between 8:1 and 6:1. In the instance where the aldehyde is glutaraldehyde and the amino acid containing species is L-glutamic acid, glutaraldehyde is optionally present from 2 to 40 weight percent of the solution with the amino acid containing species being introduced in an amount to satisfy the recited ratio. As with cross-linkable protein solution, the cross-linking agent solution optionally includes pH modifiers, surfactants, antioxidants, osmotic agents and preservatives. Examples of pH modifiers include acetic acid, boric acid, hydrochloric acid, sodium acetate, sodium bisulfate, sodium borate, sodium bicarbonate, sodium citrate, sodium hydroxide, sodium nitrate, sodium phosphate, sodium sulfite, and sulfuric acid. Surfactants operative herein illustratively include benzalkonium chloride. Antioxidants operative herein illustratively include bisulfates. Electrolytes operative herein illustratively include sodium chloride. Preservatives operative herein illustratively include chlorobutanol, sorbate, benzalkonium chloride, parabens, and chlorhexadines.

A tissue adhesive optionally includes a radioopaque material so that an inventive process optionally includes subjecting the area of a malignancy to X-ray, nuclear magnetic resonance, or positron emission topography to determine the localization of the adhesive relative to a tumor or to normal tissue. Illustrative examples of radioopaque materials illustrative include: barium salts such as barium sulfate, optionally halogenated barium salts, other heavy metal salts illustratively salts of bismuth, silver or lead; heavy metals embedded in silica filler added to the adhesive composition; organic iodine compounds illustratively methacylates; or halogenated polymers. Illustrative examples of radioopaque materials are illustratively found in U.S. Pat. No. 4,866,132 and reference cited therein.

Methods of producing a tissue adhesive are illustrated in U.S. Application Publication No. 2009/0287313.

The tissue adhesive regardless of specific formulation is applied to overlie a suspect malignancy or onto a structure with the suspect malignancy in or within 2 cm of the structure then being brought into contact with the adhesive intermediate therebetween. The tissue adhesive when a two-part formulation is used is readily applied with a proportional sized double-barreled syringe equipped with a mixing tip while one-part adhesives are readily applied through a tube or single-barreled syringe. Alternatively, a mixing tip is replaced by a spray nozzle tip to provide an atomized spray of adhesive.

A tissue adhesive composition is alternatively delivered to a site of application as a three-component system including cross-linking agent, cross-linkable protein, and a structure formed of a structure material that is a patch material. Collagen is exemplary of structure materials used herein. Alternatively, transplanted or autologous tissue such as pericardial tissue may also be used. The structure material is optionally formed as an aqueous suspension that is delivered prior to, or in concert with, an inventive cross-linking agent component and a cross-linkable protein component. Simultaneous delivery of a structure material is facilitated by the use of a three-barreled syringe where the first and second barrels deliver cross-linkable protein and cross-linking agent as detailed above and the third barrel is loaded with structure material. Optionally, a mixing tip is provided with a triple-barreled syringe. Alternatively, a structure material suspension is intermixed with the cross-linkable protein component according to the present invention and delivered as a two-component system by way of a mixing or spray nozzle tip as detailed hereinabove.

Optionally, a foaming agent is introduced into an adhesive component to facilitate the formation of a foamed tissue adhesive. A foaming agent operative herein includes tissue compatible surfactants. Illustrative of these foaming agents are non-toxic surfactants including, but are not limited to, fats or proteins in edible foams. However, the surfactant may be an ionic or non-ionic surfactant depending on the intended application. The ionic surfactants including, for example, anionic surfactants such as sodium stearate, sodium dodecyl sulfate, α-olefinsulfonate and sulfoalkylamides and cationic surfactants such as alkyldimethylbenzylammonium salts, alkyltrimethylammonium salts and alkylpyridinium salts; and amphoteric surfactants such as imidazoline surfactants. The non-ionic surfactants including, for example, polyethylene oxide alkyl ethers, polyethylene oxide alkylphenyl ethers, glycerol fatty acid esters, sorbitan fatty acid esters, sucrose fatty acid esters, and the like.

In situations where the tissue adhesive composition is delivered in conjunction with a foaming agent, a propellant is optionally provided in fluid communication with a spray nozzle tip. Propellants operative herein illustratively include aerosol propellants such as carbon dioxide, nitrogen, propane, fluorocarbons, dimethyl ether, hydrochlorofluorocarbon-22, 1-chloro-1,1-difluoroethane, 1,1-difluoroethane, and 1,1,1-trifluoro-2-fluoroethane, alone or in combination.

A pharmaceutical agent is optionally included in one-part tissue adhesive, one or both parts of a two-part tissue adhesive, or impregnated into a structure so adhesed proximal to a suspect malignancy. A pharmaceutical agent operative herein illustratively includes an analgesic, an anesthetic, an anthelminthic, an anti-allergic, an anti-arrhythmic, an anti-asthmatic, an antibiotic, an anticonvulsant, an antidepressant, an anti-diabetic, an antifungal, an antihypertensive, an anti-inflammatory agent, anti-migraine, an anti-neoplastic, an anti-parasitic, an anti-tumor agent, an anti-ulcer agent, an antiviral, an anxiolytic, a bronchodilator, a cough or cold agent, a cytostatic, a hypnotic, a hypoglycemic, a metastasis inhibitor, a muscle relaxant, a neoplastic, a sedative and a tranquilizer compound. Remington's Pharmaceutical Sciences, 16th Ed., 1980, Mack Publishing Co., Easton, Pa. and in Goodman and Gilman's The Pharmacological Basis of Therapeutics by Hardman and Limbird, 9th Ed., 1996, McGraw-Hill, New York and in The Merck Index: an encyclopedia of chemicals, drugs, and biologicals, 12th Edition, 1996, Merck & Co., Whitehouse Station, N.J.

Pharmaceutical agents deliverable by the present invention are those with a molecular weight in the range from about 50 Daltons to about 10,000,000 Daltons.

Prodrugs are included in the present invention as pharmaceutical agents. The term “prodrug” refers to compounds that are rapidly transformed in vivo to yield the parent compound of the above formula, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

In addition, it is intended that the present invention include compounds made either using standard organic synthetic techniques, including combinatorial chemistry or by biological methods, such as through metabolism.

The compositions optionally include an effective amount of the selected pharmaceutical agent or agents in combination with a pharmaceutically acceptable carrier and, in addition, may include other medicinal agents, pharmaceutical agents, carriers, or diluents. By “pharmaceutically acceptable” is meant a material that is not biologically or otherwise undesirable, which can be administered to an individual along with the selected substrate without causing significant undesirable biological effects or interacting in a deleterious manner with any of the other components of the pharmaceutical composition in which it is contained.

A single pharmaceutical agent is delivered by the drug delivery device of the present invention. Optionally, two or more pharmaceutical agents may be delivered simultaneously by the drug delivery device of the present invention.

A “structure” as defined herein is a sheet, ring, plug or dam having sufficient thickness to serve as a barrier against malignant cell transference therethrough to serve as tensioning point or manipulation of the underlying tissue to facilitate excision of an underlying suspected malignancy. Inventive structure materials optionally include collagen; polylactic acid; hyaluronic acid; fluoropolymers; thermoplastics; silicones; knitted or woven meshes of, for example, cellulosic fibers, polyamides, rayon acetates and titanium; polypropylene; polyester; skin; bone; titanium and stainless steel. Collagen is an illustrative structure material. Alternatively, pericardial or other body tissue may be used instead of a collagen patch. Optionally, the collagen is a flexible, fibrous sheet readily formed into a variety of shapes that is bioabsorbable and has a thickness of 2-5 millimeters. Such fibrous sheet collagen is commercially available from a number of suppliers. A collagen patch serves to enhance adhesive strength while allowing some penetration of the tissue sealant into the structure. Optionally, in a surgical setting, a dry or a wetted absorbent gauze is placed proximal to the wound site in order to wick away any excess ungelled inventive tissue sealant prior to cure.

A structure is optionally placed in direct contact with a tumor or within a specified distance of a tumor with the distance being traversed by non-malignant tissue. A distance is optionally less than 2 centimeters, optionally less than 1, 0.9, 0.8, 0.7, 0.6, 0.5, 0.4, 0.3, 0.2, 0.1, 0.01, or less centimeters, optionally zero centimeters. It is appreciated that a distance is defined as the shortest distance between any point on a tumor and any point on a structure.

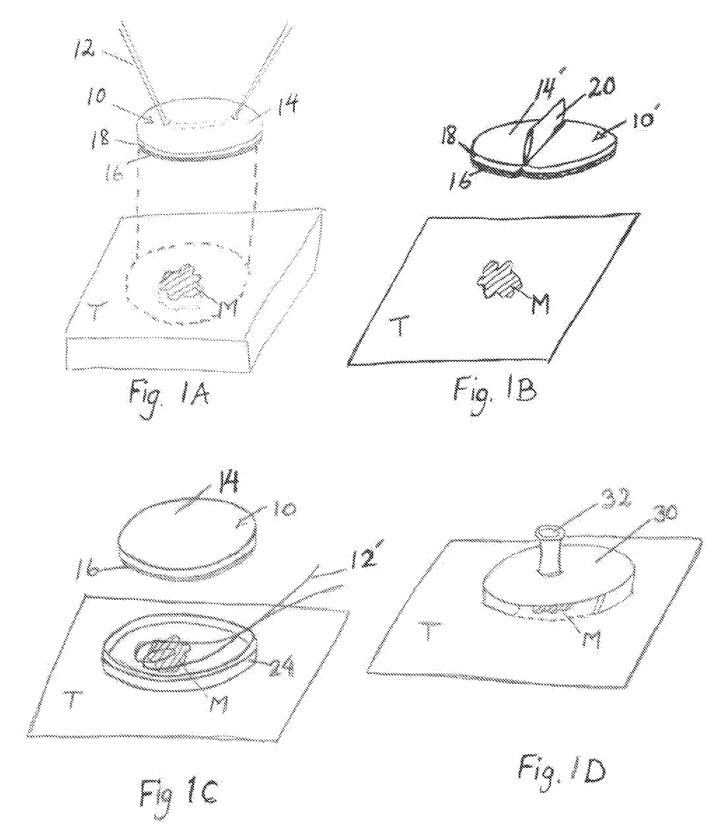

Referring now to FIGS. 1A-1D where like numerals used between figures have a like meaning, it is noted that the relative dimensions of features depicted therein are distorted for visual clarity. The structure 10 is shown in FIG. 1A projected above a region of tissue T inclusive of a possible malignancy M. The structure 10 has a suture 12 extending from an exposed surface 14 of the structure 10. The suture 12 serves as a tensioning point used to draw the malignancy M upward or sideways to facilitate resection. A tissue adhesive 16 is applied either directly to the malignancy M or alternatively onto an opposing surface 18 of the structure 10. Optionally, the structure 10 and the adhesive 16 are dimensioned and applied, respectively, to define a desired margin around the malignancy M and into surrounding tissue. A margin is optionally 2 centimeters or less, optionally less than 1, 0.9, 0.8, 0.7, 0.6, 0.5, 0.4, 0.3, 0.2, 0.1, 0.01, or less centimeters. In some embodiments, a margin is between 1 and 5 millimeters beyond the extent of the malignancy M. In FIG. 1B, a structure 10′ is creased to form a flap 20 extending from the surface 14′ as an alternative tensioning point. A layer of adhesive 16 is applied intermediate between the opposing surface 18 and a target malignancy M.

FIG. 1C depicts an inventive first structure 10 with adhesive 16 applied intermediate between opposing surface 18 and an underlying malignancy M with a wire, polymeric, or other suture material known in the art illustratively sheep intestine, attachment 12′ positioned intermediate between the malignancy M and the opposing surface 18. A second structure in the form of a dam 24 defines the boundaries of spread of adhesive 16 upon overlying contact with a malignancy M. The dam 24 also provides a strengthening surface when formed of a material to which the adhesive 16 also adheres. In surgical practice, resection around the outer boundary of the dam 24 as part of removal of a malignancy that with tensioning of attachment 12′ allows for more rapid and efficient malignancy removal while inhibiting malignancy spread.

A dam is optionally formed of any material to which an adhesive will adhere. Optionally, a dam is formed of a material to which an adhesive will not adhere. Optionally, a dam and a first structure are optionally formed of the same material, optionally with the same or different rigidity. A dam optionally has increased rigidity relative to a first structure so as to define a substantially rigid boundary around which to place or guide a cutting instrument to resect the tumor. In some embodiments, a dam is an inflatable or otherwise expandable structure that is optionally positioned in a deflated state and is then inflated to serve as a barrier for the prevention of tissue adhesive flow prior to cure. Once the adhesive is adhered or cured, the balloon is optionally deflated and removed.

FIG. 1D depicts in partial cutaway a casting form 30 sized to overlay a malignancy M and define a boundary thereabout. The casting form 30 includes a port 32 through which a liquid tissue adhesive is inserted. Optionally, the port 32 is configured as a luer lock adapted to receive a complementary syringe fitting. With insertion of adhesive 16 into the casting form 30, the form is held in position until the adhesive cures and the casting form is adhered in place and surgically resected along with an underlying malignancy M. A casting form 30 is readily formed from a variety of materials such as those from which an inventive structure is formed. A casting is optionally used alone or in conjunction with an intermediate structure.

Any superficial or solid deep tissue tumor may be resected by an inventive process. Illustrative superficial tumor types include epithelial tumors such as tumors of the head, neck, colon, bladder or other urinary tract position, lung, cervix, among others known in the art. Deep tissue tumors are optionally resected by inventive processes. Resection of a deep tissue tumor includes one or more incisions to expose a surface area of normal tissue adjacent to a tumor or the tumor tissue itself prior to administration of a tissue adhesive and/or a structure. The structure is adhered to the tissue by an adhesive whereby the structure alone or in conjunction with a second structure such as a dam define a cutting region for subsequent tumor resection.

It is appreciated that the structure allows a surgeon to apply excisive pressure to a tumor or surrounding tissue to more easily expose normal tissue upon which to cut and reduce the risk of inadvertent cutting into a lower region of a tumor. A structure when adhered to tissue, therefore, optionally serves to expose normal tissue below a tumor for cutting thereby improving outcome and reducing metastases by contact of a surgical instrument with a tumor cell.

Also provided are processes for inhibiting malignant cell transference during a biopsy procedure. Processes optionally include applying a tissue adhesive to a surface of a flexible structure, and placing the flexible structure into a track formed by a biopsy needle into a suspected malignancy.

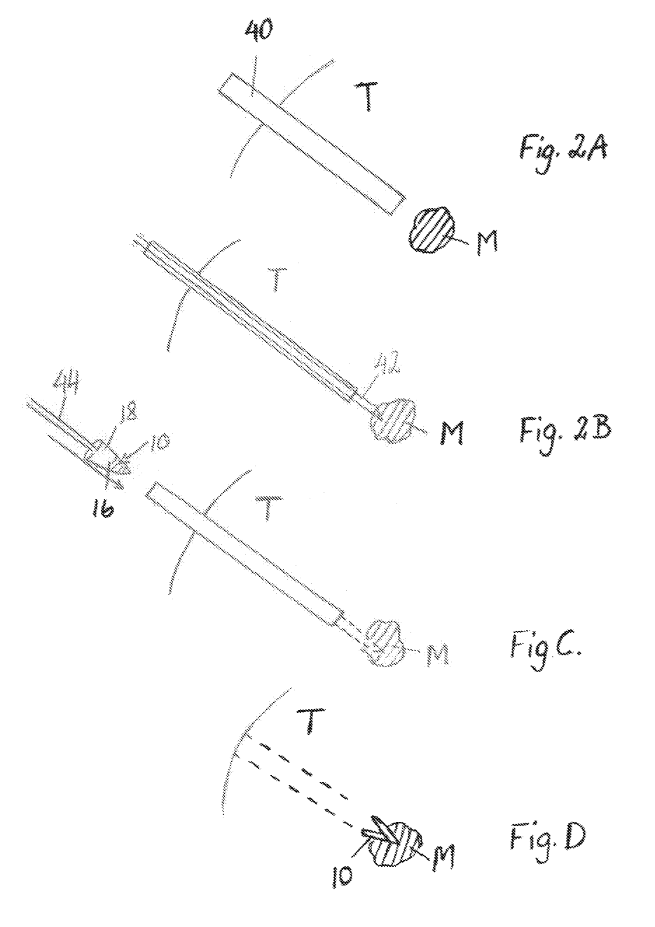

FIGS. 2A-2D depict processes according to some embodiments by which an inventive structure inhibits malignant cell transference in the course of a biopsy procedure. In FIG. 2A, a cannula 40 penetrates normal tissue T but not into contact with suspect malignancy M. As shown in FIG. 2B, as is conventional to a biopsy procedure, a biopsy needle 42 is passed through the cannula 40 and into the malignancy M to secure a tissue sample thereof. With the withdrawal of the biopsy needle 42, malignant cells are contacted with flowing blood associated with the track created by the biopsy needle 42 through the malignancy M and surrounding normal tissue T. In FIG. 2C, tissue adhesive, optionally applied to an opposing surface 18 of a flexible wadding of a structure 10, is pushed through the cannula 40 prior to adhesive cure. If a structure 10 is present, the structure 10 is optionally inserted with a ram 44, otherwise a syringe barrel is well suited for delivery of a liquid or gelatinous adhesive alone. The adhesive alone or in conjunction with a structure 10 forms an adhesed plug corresponding to the track of the biopsy needle 42 beyond the cannula 40 thereby inhibiting malignant cell transfer through the bloodstream or surrounding tissues.

A biopsy needle 42 or cannula 40 optionally includes a closing device in the form of a balloon or other closing device that serves to prevent exposure of normal tissue to malignant tumor tissue either during or after removal of a biopsy needle. A balloon or other closing instrument is optionally detachable from the biopsy instrument. Processes optionally include retrieving a sample of tumor tissue with a biopsy instrument and inflation of a balloon or other closing instrument to form a physical barrier that expands alone with the removal of the biopsy instrument preventing exposure of a tumor cell to the bloodstream or surrounding normal tissue. A balloon is optionally formed of structure material that is capable of being wetted by adhesive. As such, an adhesive applied during or following deployment of a balloon wets the balloon from the side opposite the tissue and by permeating the structure contacts the tissue forming an adhesive plug supported by the structure. Wetting is optionally enhanced by pressure applied to the adhesive during application essentially forcing adhesive into and through the closing instrument.

In some embodiments, one or more barrels of an adhesive delivery device are inserted into a cannula or are positioned integral with a cannula or needle. Optionally, the barrels are external to a biopsy instrument channel within the cannula or within the biopsy instrument channel so that adhesive materials or precursors may be presented within normal tissue simultaneously with the presence of a biopsy instrument. Following attachment a sample of tumor tissue, a biopsy needle is withdrawn back toward the cannula. In some embodiments, when the biopsy needle is positioned within the distal end of the cannula, adhesive is injected into the track left by the biopsy needle. This initial deposition of adhesive optionally provides a first barrier against tumor cell migration and may serve to physically push free tumor cells down into the tumor tissue that otherwise would have sufficient time to associate with normal tissue or enter the blood stream. The adhesive is optionally applied within 10 seconds of the tip of a biopsy instrument entering a cannula, optionally within 9, 8, 7, 6, 5, 4, 3, 2, 1 or fewer seconds. Optionally, adhesive is applied simultaneous with retraction of the biopsy instrument from the malignancy.

In some embodiments, a flexible structure is then inserted into the cannula through the biopsy instrument channel to adhere to the delivered adhesive to form a structure supported adhered plug corresponding to shape of the track of the biopsy needle 42 beyond the cannula 40 and thereby inhibiting malignant cell transfer through the bloodstream or surrounding tissues. Additional adhesive is optionally applied to a structure prior to its insertion into the channel to improve the adhesive bond to the previously injected adhesive or to supplement the previously injected adhesive. The structure is optionally inserted prior to or subsequent to curing of the initially injected adhesive. A structure is optionally positioned in the track within one minute of removal of a biopsy instrument, optionally within 30, 20, 10, 5, 4, 3, 2 or fewer seconds of removal of the biopsy instrument.

In embodiments that include one or more barrels of an adhesive delivery device integral with a cannula, that these barrels function similar to a syringe as described herein and are optionally equipped with a mixing tip. Alternatively, a mixing tip is replaced by a spray nozzle tip to provide an atomized spray of adhesive. One or more barrels each individually containing a component of an adhesive optionally combine to a single orifice optionally including a mixing tip or nozzle tip such that mixing of the individual components optionally occur simultaneous with their administration to the track left by the biopsy instrument.

Various modifications of the present invention, in addition to those shown and described herein, will be apparent to those skilled in the art of the above description. Such modifications are also intended to fall within the scope of the appended claims.

It is appreciated that all reagents are obtainable by sources known in the art unless otherwise specified. Methods of nucleotide amplification, cell transfection, and protein expression and purification are similarly within the level of skill in the art.

Patents and publications mentioned in the specification are indicative of the levels of those skilled in the art to which the invention pertains. These patents and publications are incorporated herein by reference to the same extent as if each individual application or publication was specifically and individually incorporated herein by reference for the entirety of their teaching.

The foregoing description is illustrative of particular embodiments of the invention, but is not meant to be a limitation upon the practice thereof. The following claims, including all equivalents thereof, are intended to define the scope of the invention.

1. A process for malignancy removal comprising:
applying a tissue adhesive in simultaneous contact between tissue and a surface of an overlying structure wherein said tissue is malignant tissue or non-malignant tissue within 2 centimeters of a malignancy;
allowing sufficient time for said adhesive to cure forming a bond between said structure and said tissue; and
cutting into a margin of normal tissue surrounding the malignancy outside said structure.

2. The process of claim 1 further comprising
providing an attachment for tensioning said structure during cutting.

3. The process of claim 2 wherein said attachment is a suture.

4. The process of claim 2 wherein said attachment is a flap formed by a crease in said structure.

5. The process of claim 1 further comprising
positioning a dam around the area of said malignancy prior to applying said adhesive.

6. The process of claim 1 wherein said structure is a casting form.

7. The process of claims 1 wherein said adhesive is a two-part adhesive comprising:
a cross-linkable protein; and
a cross-linking agent solution comprising an aldehyde and an amino acid containing species reactive with said aldehyde, said aldehyde and said amino acid containing species being present in a ratio between 20:1 and 1:1 and said protein and said cross-linking agent are present in a ratio of between 15:1 and 1:1.

8. The process of claim 1 wherein said structure comprises collagen.

9. The process of claim 8 wherein said collagen is in the form of a sheet.

10. The process of claim 1 wherein said adhesive, said structure, or both includes a pharmaceutical agent.

11. A process for inhibiting malignant cell transference during a biopsy procedure comprising:
applying a first tissue adhesive to a track formed by a biopsy needle.

12. The process of claim 11 further comprising:
placing a flexible structure into said track in contact with said adhesive to form a structure supported adhesive plug.

13. The process of claim 12 wherein further comprising
applying a second tissue adhesive to said structure prior to said placing.

14. The process of claim 12 further comprising
applying a second adhesive to said structure and subsequently placing said structure into said track and in contact with said first adhesive to form a structure supported adhesive plug.

15. The process of claim 11 wherein said applying is within 10 seconds of a biopsy needle retracting into a cannula.

16. The process of claim 11 wherein said applying is to a closing instrument detachable from a biopsy needle.

17. The process of claim 11 wherein said first adhesive is a two-part adhesive comprising:
a cross-linkable protein; and
a cross-linking agent solution comprising an aldehyde and an amino acid containing species reactive with said aldehyde, said aldehyde and said amino acid containing species being present in a ratio between 20:1 and 1:1 and said protein and said cross-linking agent are present in a ratio of between 15:1 and 1:1.

18. The process of claim 14 wherein said first adhesive and said second adhesive are compositionally identical.

19. The process of claim 11 wherein said first adhesive or said structure includes one or more pharmaceutical agents.

20. (canceled)

21. The process of claim 11 wherein said first adhesive is administered from a cannula with adhesive delivery channels integral therewith or physically associated therewith.
